(19) United States
(12) Patent Application Publication
(10) Pub. No.: US 2025/0170196 A1
(43) Pub. Date: May 29, 2025

(54) ANTI-INFLAMMATORY, IMMUNOMODULATORY AND ANALGESIC AGENTS FROM TREE EXUDATES

(71) Applicant: LivFul Inc., Alpharetta, GA (US)

(72) Inventor: Luiz Francisco PIANOWSKI, ITATIBA (BR)

(21) Appl. No.: 18/836,544

(22) PCT Filed: Feb. 7, 2023
(86) PCT No.: PCT/BR2023/050042

§ 371 Date: Aug. 7, 2024

Related U.S. Application Data
(60) Provisional application No. 63/307,344, filed on Feb. 7, 2022.

(51) Int. Cl. A61K 36/328; A61K 36/324; A61P 35/00

(52) U.S. Cl. CPC A61K 36/328; A61K 36/324; A61P 35/00; A61K 2236/33

(57) ABSTRACT

The invention refers to the use resin extracts of tree species, related to Boswellia carterii, Boswellia negletta, Commiphora holziana, in treating chronic inflammation conditions, potentially leading to cancer, or autoimmune diseases like Alzheimer, Crohn's syndrome, psoriasis, multiple sclerosis, etc., and in relieving pain. A key aspect of our invention is the selection of the resin extracts or their components, able to modulate protein kinases activity, including PKC isoforms activity. The invention also relates to a method for producing extracts with a high concentration of bioactive compounds.

### FIELD OF THE INVENTION

The invention refers to the use resin extracts of tree species, related to Boswellia carterii, Boswellia negletta, Commiphora holziana, in treating chronic inflammation conditions, potentially leading to cancer, or autoimmune diseases like Alzheimer, Crohn's syndrome, psoriasis, multiple sclerosis, etc., and in relieving pain. A key aspect of our invention is the selection of the resin extracts or their components, able to modulate protein kinases activity, including PKC isoforms activity. The invention also relates to a method for producing extracts with a high concentration of bioactive compounds.

### STATE OF THE ART

Oleoresin from species of Boswellia, commonly called frankincense, is a rich source of terpenoids with a broad spectrum of therapeutic applications. Boswellic acids and their derivatives, common components of the resin, are the most known for their health benefits. However, the oleoresins from different sources (species and location) display wide variability in composition of terpenes, and therefore may display different modes of bioactivity in people and animals due to specific components concentration or a specific synergy between these components. Other known compounds in such exudates are derivatives of tirucallic acids, amyrins, lupeol derivatives, incensole just to mention a few.

It is evident from current research that to evaluate the desired therapeutic effect of such extracts a plethora of methods for biological screening are available, usually based on markers of particular pathological conditions or on anti-proliferation test in vitro. It is imperative to accelerate drug discovery by choosing more general screening method, especially dealing with complex materials from plants.

There are documented effects of terpenoids on tumors growth and cells apoptosis and strong connection between protein kinases PKC and the observed effects. For example, inhibition of pancreatic tumor by dietary farnesol and geraniol is involving PKC signaling system.

### DESCRIPTION OF INVENTION

### Extraction Process

The extraction process was focused on a fast and full recovery of soluble components of oleoresin in hexanes, water and/or ethanol. According to the present invention, the extraction process also encompasses dichloromethane (DCM) or supercritical extractions.

The resin was frozen to −40 deg and micronized in 1 minute in a high speed mill. The powdered resin was first extracted twice with hexanes at room temperature in sealed vessels. The clear supernatants were decanted, combined and evaporated at 35 deg under vacuum. Our concern was to minimize exposure the material to air and light at elevated temperatures.

Dry residue after hexanes extraction was mixed with water to solubilize polar components of the resin. The gummy residue was collected on sintered glass funnel, and aqueous filtrate evaporated under vacuum.

Remaining semi-dry residue was subjected to ethanol extraction for 24 hrs. The solids and supernatant were separated by filtration on sintered glass funnel, and the filtrate was evaporated under vacuum at 40 deg to obtain glassy solids.

### Isolation of Acids From Hexane Fraction

For acidic terpenes isolation we used aqueous ammonia instead 1M NaOH solution to minimize de-acylation. Acidic terpenes precipitate easily in hexanes after ammonia treatment. The acid-depleted hexane solution is removed by decantation, and the precipitate was further washed with a fresh portion of hexanes. The ammonia salts were partitioned between ethyl acetate and 1M HCl to obtain solid acidic terpenes mixture after evaporation of the organic solvent.

### Oleoresin Compounds

Typical compounds found in oleoresin from Boswellia are:
boswellic acids
keto-boswellic acids
tirucallic acids
lupeolic acids
serratol
roburic acids
incensole (B. negletta)
caryophyllene oxide
canaric acid (B. negletta)
amyrins (B. negletta)

Typical compounds found in Commimophora are:
germacrone
furanoterpenoids
lupenyl compounds
amirins
curzerenone
guggulsterones
guggulsterols

### Biological Screening Method for Anti-Inflammatory and analgesic Action Using PKC Assays

PKC isozymes can act as therapeutic targets as they are involved in multiple signal transduction systems that respond to a variety of external stimulators, including hormones, growth factors, and other membrane receptor ligands.

In cancer cells, PKC isozymes are involved in cell proliferation, survival, invasion, migration, apoptosis, angiogenesis, and anticancer drug resistance through their increased or decreased participation in various cellular signaling pathways.

Several PKC isoenzymes have also been shown to be important mediators in inflammation and immunity, particularly in lymphocyte responses.

PKC isozymes have been found in the anatomical areas that regulate pain and nociception, and there is growing evidence that PKC is involved in isozyme-specific effects in pain modulation.

Inhibition or impairment of PKC activity results in compromised learning and memory, whereas an appropriate activation of certain PKC isozymes leads to an enhancement of learning and memory. In preclinical studies, PKC activators have been shown to increase the expression and activity of PKC isozymes, thereby restoring PKC signaling and downstream activity, including stimulation of neurotrophic activity, synaptic/structural remodeling, and synaptogenesis in the hippocampus and related cortical areas. PKC activators also reduce the accumulation of neurotoxic amyloid and tau protein hyperphosphorylation and support anti-apoptotic processes in the brain. These observations strongly suggest that PKC pharmacology may represent an attractive area for the development of effective cognition-enhancing therapeutics for the treatment of dementias.

In an embodiment, the present invention is related to the mixture of Commiphora myrrha—myrrh (95%) and Boswellia sacra—frankincense (5%).

According to the present invention, it was verified an inhibition of cytokines, supporting the treatment of COVID-19.

These extracts are obtained through extraction with DCM Dichloromethane and subsequent total removal of the solvent until monoterpenes begin to be released. With this procedure to increase to a temperature above the boiling point of DCM we are certain of its total elimination.

### EXAMPLES

### Evaluation of the Cytotoxicity of Plant Extract in Different Tumor Cells

### Cytotoxicity Activity of ST-160.5 at Different Concentrations by MTT Assay

Cells were examined by MTT-assay to determine the ST-160.5 induced cytotoxicity. Myrrh Resin extract (ST-160.5) exposure showed concentration-dependent cytotoxicity for five cell lines studied after 24 hours of incubation (FIG. 2). The viability of the A549 cell (lung cancer—FIG. 2A) was decreased to 42.54% and 9.69% in concentration of 100 and 300 μg/mL, respectively at 24 hours with mean IC50:82.88 μg/mL (57.06 to 121.9). In Caco-2 cell line (colorectal adenocarcinoma—FIG. 2B), cell viability was decreased to 57.42% and 15.79% at concentration of 100 and 300 μg/mL, respectively at 24 hours with mean IC50:120.1 μg/mL (65.25 to 232.7). The viability of the MDA-MB-231 cells (breast adenocarcinoma—FIG. 2C) was decreased to 47.43% and 13.68% in concentration of 100 and 300 μg/mL, respectively at 24 hours with mean IC50:76.16 μg/mL (47.53 to 124.1). In the case of SH-4 cells (melanoma—FIG. 2D), cell viability was decreased to 40.57% and 12.58% in concentration of 100 and 300 μg/mL, respectively at 24 hours with mean IC50:90.34 μg/mL (49.61 to 173.3). In THP-1 cells (Acute monocytic leukemia—FIG. 2E) cell viability was decreased to 79.46% and 17.3 2% in concentration of 30 and 100 μg/mL, respectively at 24 hours with mean IC50:52.36 μg/mL (29.67 to 94.64).

Figure: Viability assessment for A549, Caco-2, MDA-MB-231, SH-4 and THP-1 cells after incubation with Test Item ST-160.5. Five types of cells were treated with ST-160.5 (1-300 μg/mL) for 24 hours. The cell viability was measured by MTT assay. Values are expressed as mean±standard error deviation of the mean of three assays in triplicate. (A) A549 cell line (green) IC50=82.88 μg/mL. (B) Caco-2 cell line (yellow) IC50=120.1 g/mL, (C) MDA-MB-231 cell line (orange) IC50=76.16 μg/mL, (D) SH-4 cell line (blue) IC50=90.34 μg/mL and (E) THP-1 cell line (red) IC50=52.36 μg/mL.

### Percentage of Viability was Calculated in Relation to the Vehicle Group (Culture Medium DMEM or RPMI1640 with 0.5% DMSO)

Results of present study demonstrate that ST-160.5 has potential cytotoxic activity on human lung cancer (A549), colorectal adenocarcinoma (Caco-2), breast adenocarcinoma (MDA-MB-231), melanoma (SH-4) and acute monocytic leukemia (THP-1) cells, indicating the presence of cytotoxic compounds in the extract.

### Evaluation of the Cytotoxicity of Plant Extracts in Different Tumor Cells

In vitro cytotoxicity testing provides a crucial means for safety assessment and screening of potential anticancer compounds. The aim of this study was to evaluate the in vitro cytotoxic activity of four plant extracts (ST-160.1, ST-160.2, ST-160.3 and ST-160.4). Different concentrations of the compounds were tested against MDA-MB-231 (breast adenocarcinoma), A549 (lung carcinoma), Caco-2 (Colorectal adenocarcinoma) and THP-1 (Acute monocytic leukemia) human cancer cell lines in order to determine the mean IC50 (50% of inhibition of cells growth) by using the MTT assay.

The cells were treated with the following concentrations of plant extracts: 1, 10, 30 and 100 μg/mL for 24 hours. Dilution from stock solutions were made in culture medium yielding final extracts concentrations with a final DMSO concentration of 1%. DMSO concentration did not affect cell viability. The compounds ST-160.1, ST-160.2, ST-160.3 and ST-160.4 showed the mean IC50 values greater than 100 μg/mL in cell lines A549 and Caco-2. On the other hand, ST-160.1 showed the mean IC50 of 39.67 μg/mL with confidence interval (CI) of 27.15 to 58.26 in MDA-MB-231 cells and mean IC50 of 41.10 μg/mL (CI of 22.74 to 75.07 in THP-1 cell). Test Item ST-160.2 presented the mean IC50 of 41.42 μg/mL (CI of 29.88 to 57.68 in MDA-MB-231 cells) and the mean IC50 of 35.00 μg/mL (CI of 21.09 to 58.09 in THP-1 cell). The ST-160.4 had the mean IC50 of 57.74 μg/mL (CI of 39.39 to 85.89 in MDA-MB-231 cells) and mean IC50 of 56.00 μg/mL (CI of 35.96 to 88.91 in THP-1 cell). Overall, this study demonstrated the cytotoxic potential of ST-160.1, ST-160.3 and ST-160.4 on human breast adenocarcinoma (MDA-MB-231) and acute monocytic leukemia (THP-1) cells, indicating the presence of cytotoxic secondary metabolites in these extracts.

Results of the present study demonstrate that ST-160.1, ST-160.3 and ST-160.4 have potential cytotoxic activity on human breast adenocarcinoma (MDA-MB-231) and acute monocytic leukemia (THP-1) cells, indicating the presence of cytotoxic secondary metabolites in these extracts.

### Exploratory Toxicity of Repeated Doses for 28 Days for Test Item 160 (st 160) in Rats

Aims: The present study aimed to evaluate the exploratory oral toxicity of Test Item 160 (ST-160) through a repeated dose for 28 days in rats.
Animals and treatments: male and female rats (5 male and 5 female per sex/group) were treated orally with Vehicle (10% Tween 80 mixed with water) or with ST-160 (60 mg/kg) once daily for 28 days.

During the treatment animals were evaluated daily for general clinical signs, morbidity and mortality and weekly for detailed clinical signs and changes in body weight. On the 29th day, animals were euthanized and necropsy was performed which included blood collection for hematological and clinical chemistry analysis followed by the collection of liver, spleen and kidneys for histopathological analysis.

### Main Results

1. The repeated oral treatment with ST-160 for 28 days did not cause morbidity nor mortality in animals.
2. Repeated treatment with ST-160 caused an increase in absolute and relative spleen weight (male).

The absolute and relative spleen weight (female), absolute and relative liver weight (male and female) and absolute and relative kidneys weight (male and female) were not changed when compared with animals of vehicle groups.
3. In addition, repeated treatment with ST-160 caused changes in some clinical chemistry parameters, such as an increase in urea serum levels and reduces total cholesterol and fasting triglycerides in male rats when compared to the vehicle group. In female, the treatment with ST-160 induces an increase in total bilirubin serum levels when compared with vehicle (female).
4. Regarding the hematological analysis, repeated treatment with ST-160 did not cause changes in all the evaluated parameters.
5. The histopathological analysis did not show alterations in liver, spleen and kidneys.

The repeated oral treatment with ST-160 for 28 days caused; i) increase in absolute and relative spleen weights in males; ii) increase in urea and decrease in total cholesterol and fasting triglycerides serum levels in male rats and an increase in total bilirubin in female rats; iii) no histopathological changes were observed in the kidneys, liver and spleen fragments.

1-6. (canceled)

7. An extract of resin from species of Boswellia and Commiphora, with a strong modulatory action on protein kinases activity that is therapeutically active against a condition selected from the group consisting of cancer, endometriosis, inflammation, pain and autoimmune disease.

8. A heath supplement comprising the extract of claim 7.

9. A method of treatment of a condition selected from the group consisting of cancer, endometriosis, inflammation, pain and autoimmune disease comprising administering an effective amount of the extract of claim 7 to a patient in need thereof.

10. The method of claim 9 wherein a plurality of the conditions are treated at the same time.

11. A method of treatment of a condition selected from the group consisting of cancer, inflammation, pain, autoimmune disease, Crohn's syndrome, psoriasis, multiple sclerosis, and endometriosis comprising administering an effective amount of the extract of claim 7 or sub-fractions thereof to a patient in need thereof.

12. The method of claim 11 wherein a plurality of the conditions are treated at the same time.

13. A pharmaceutical formulation comprising the extract of claim 7.

14. A pharmaceutical formulation comprising a pure extract of claim 7.

15. A pharmaceutical formulation comprising a sub-fraction of the extract of claim 7.

16. A method of enrichment of the active substances in the extract of claim 7 comprising obtaining the extract using ammonia or amine solutions.

17. An extract of resin from species of Boswellia and Commiphora, with a strong modulatory action on protein kinases activity that is therapeutically active against a condition selected from the group consisting of cancer, inflammation, pain, autoimmune disease, Crohn's syndrome, psoriasis, multiple sclerosis, and endometriosis.
